(19) United States
(12) Patent Application Publication
(10) Pub. No.: US 2010/0113608 A1
(43) Pub. Date: May 6, 2010

(54) Tryptase Enzyme Inhibiting Aminothiophenols

(76) Inventors: Randall S. Alberte, Estero, FL (US); William P. Roschek, JR., Naples, FL (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/611,239

(22) Filed: Nov. 3, 2009

Related U.S. Application Data
(60) Provisional application No. 61/111,065, filed on Nov. 4, 2008.

(51) Int. Cl. A61K 31/136; C07C 211/54; A61P 29/00; A61P 37/08

(52) U.S. Cl. 514/658; 564/433; 564/434

(57) ABSTRACT

Disclosed herein are novel compounds and pharmaceutical compositions comprising these compounds. In some embodiments, the compounds are inhibitors of the tryptase enzyme and are useful for treating allergic rhinitis, asthma, vascular injury (e.g., restenosis and atherosclerosis), inflammatory bowel disease, arthritis, psoriasis, anaphylaxis, wounds, infections, and other allergy and inflammatory related diseases.

### RELATED APPLICATIONS

This application claims the benefit of priority to U.S. Provisional Application No. 61/111,065, filed on Nov. 4, 2008, the contents of which are hereby incorporated in their entirety.

### FIELD OF INVENTION

The present invention relates to small molecule inhibitors of the tryptase enzyme that are useful for treating allergic rhinitis, asthma, vascular injury (e.g., restenosis and atherosclerosis), inflammatory bowel disease, arthritis, psoriasis, anaphylaxis, wounds, infections, and other allergy and inflammatory related diseases.

### BACKGROUND OF THE INVENTION

Tryptase is a tetrameric serine protease with a molecular size of 134 kD comprised of four monomers of 32-34 kD, each with one catalytic site (C. P. Sommerhoff, W. Bode, P. J. Pereira, M. T. Stubbs, J. Sturzebecher, G. P. Piechottka, G. Matschiner and A. Bergner, 1999. The structure of the human betaII-tryptase tetramer: fo(u)r better or worse, Proc. Natl. Acad. Sci. USA. 96:10984-10991). Its presence is restricted almost exclusively to mast cells, where tryptase exists within the secretory granules in a complex with heparin proteoglycan (J. A. Cairns and A. F. Wells, 1997. Mast cell tryptase stimulates the synthesis of type I collagen in human lung fibroblasts, J. Clin. Invest. 99:1313-1321). Mast cells are becoming distinguished as essential sources of inflammatory cytokines, including interleukins 1, 4 and 6, tumor necrosis factor, transforming growth factor, and basic fibroblast growth factor which may have roles in controlling processes of inflammation and fibrosis (J. A. Cairns and A. F. Wells, 1997. Mast cell tryptase stimulates the synthesis of type I collagen in human lung fibroblasts, J. Clin. Invest. 99:1313-1321).

Key control points in allergic rhinitis, an inflammatory response to particulates like pollen, dust and related allergens, include the enzymes that control the flow of arachidonic acid into an inflammatory cascade that generates prostaglandins and leukotrienes. The major players in the cascade are histamine production and release (H₁ receptors), prostaglandin D₂ Synthase responsible for the production of certain pro-inflammatory prostaglandins, the Leukotriene Receptor that controls pro-inflammatory leukotriene release, and Tryptase. Tryptase, in particular, controls the degranulation of Mast cells and certain Basophils that that contain a broad diversity of cytokines and chemokines that drive the inflammatory manifestation of allergic rhinitis; these include, runny nose, itchy and watery eyes, sneezing, itchy skin, and issue swelling (P. Edwards, 2006. Combinatorial approach towards the discovery of tryptase inhibitors, Drug Discov. Today. 11:181-182; W. Cookson, 2002. Genetics and genomics of asthma and allergic diseases, Immunol. Rev. 190:195-206; J. W. Steinke, S. S. Rich and L. Borish, 2008. Genetics of allergic disease, J. Allergy Clin. Immunol. 121:S384-S387).

Tryptase also plays a critical role in arthritis, as the presence of both major forms of tryptase in synovial fluid indicates that mast cell products are secreted constitutively, as well as by processes of anaphylactic degranulation in conditions of rheumatoid arthritis, seronegative spondyloarthritis and osteoarthritis (M. G. Buckley, C. Walters, W. M. Wong, M. I. Cawley, S. Ren, L. B. Schwartz and A. F. Walls, 1997. Mast cell activation in arthritis: detection of alpha- and beta-tryptase, histamine and eosinophil cationic protein in synovial fluid, Clin. Sci. (Lond.). 93:363-370). More recently it has been shown that intra-articular injection of (3-tryptase results in rapid joint swelling in wild-type mice that was completely abrogated in PAR-2^{-/-} mice, suggesting that tryptase-mediated inflammatory actions require functional PAR-2. Tryptase plays an important role in mediating chronic inflammation as APPA co-administration substantially inhibited FCA-induced joint swelling. Therefore, PAR-2 plays a key role in mediating chronic joint inflammation and tryptase serves as a crucial activator of PAR-2-mediated actions (E. B. Kelso, L. Dunning, J. C. Lockart, W. R. Ferrell, R. Pelvin and C. P. Sommerhoff, 2005. Tryptase as a PAR-2 activator in joint inflammation, Arthrit. Res. Ther. 7:P99). Tryptase found in the synovium of rheumatoid arthritis patients was identical to human mast cell tryptase, which was composed of two subunits of 33 and 34 kDa. Mast cell tryptase activity in rheumatoid arthritis synovial fluid was significantly higher than that in osteoarthritis synovial fluid, though it was elevated in osteoarthritis patients as well (S. Nakano, T. Mishiro, S. Takahara, H. Yokoi, D. Hamada, K. Yukata, Y. Takata, T. Goto, H. Egawa, S. Yasuoka, H. Furouchi, K. Hirasaka, T. Nikawa and N. Yasui, 2007. Distinct expression of mast cell tryptase and protease activated receptor-2 in synovia of rheumatoid arthritis and osteoarthritis, Clin. Rheumatol. 26:1284-1292).

A prominent feature of chronically inflamed tissue, fibrosis, is characterized by progressive and extreme accumulation of extracellular matrix collagen as a result of increased proliferation of fibroblasts. Fibroblasts are the key mesenchymal cell accountable for the synthesis of interstitial collagen. A characteristic of lung tissue from patients with fibrotic lung disease is an elevated number of mast cells, many of which are in a state of degranulation located in close proximity to proliferating fibroblasts (J. A. Cairns and A. F. Wells, 1997. Mast cell tryptase stimulates the synthesis of type I collagen in human lung fibroblasts, J. Clin. Invest. 99:1313-1321). Also present are increased concentrations of tryptase and other mast cell products in bronchoalveolar fluid gathered from patients with fibrotic lung disease (J. A. Cairns and A. F. Wells, 1997. Mast cell tryptase stimulates the synthesis of type I collagen in human lung fibroblasts, J. Clin. Invest. 99:1313-1321). The anti-inflammatory action in the lungs would also decrease the bronchoconstriction and have anti-tussive potential. Though current research is focusing on the identification and development of tryptase inhibitors (B. J. Newhouse, 2002. Tryptase inhibitors—review of the recent patent literature, IDrugs. 5:682-688), new tryptase inhibitors are needed to treat a host of inflammatory diseases.

### SUMMARY OF THE INVENTION

The present invention relates to novel compounds and pharmaceutical compositions comprising these compounds. In one aspect, the invention relates to a substantially pure and isolated compound of formula I:

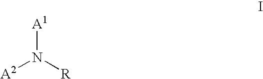

or a pharmaceutically acceptable salt thereof,
wherein, independently for each occurrence,
A¹ and A² are each aryl; and
R is alkyl, cycloalkyl, heterocycloalkyl, alkenyl, alkynyl, aryl, heteroaryl, aralkyl, or heteroaralkyl;
wherein any of the aforementioned alkyl, cycloalkyl, heterocycloalkyl, alkenyl, alkynyl, aryl, heteroaryl, aralkyl, or heteroaralkyl may be optionally substituted with one or more groups selected from the group consisting of halo, azido, alkyl, haloalkyl, aralkyl, alkenyl, alkynyl, cycloalkyl, heterocyclyl, aryl, heteroaryl, heteroaralkyl, hydroxy, alkoxy, aryloxy, heteroaryloxy, amino, nitro, sulfhydryl, imino, amido, phosphonate, phosphinate, acyl, carboxyl, oxycarbonyl, acyloxy, silyl, thioether, sulfonate, sulfonyl, sulfonamido, formyl, cyano and isocyano.

In some embodiments, A¹ is a phenyl. In certain embodiments, the phenyl is substituted with at least one of a halo, alkyl, haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹, —N(R¹¹)₂ or —N(R¹¹)C(═O)R¹¹; and R¹¹ is hydrogen, or alkyl, haloalkyl, cycloalkyl, heterocycloalkyl, alkenyl, alkynyl, aryl, heteroaryl, aralkyl, or heteroaralkyl. In other embodiments, the phenyl is substituted with, —N(R¹¹)₂ or —N(R¹¹)C(═O)R¹¹. In another embodiment, the phenyl is substituted with, —N(R¹¹)₂, wherein R¹¹ is hydrogen.

In some embodiments, A² is phenyl. In certain embodiments, the phenyl is substituted with at least one of a halo, alkyl, haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹, —N(R¹¹)₂ or —N(R¹¹)C(═O)R¹¹; and R¹¹ is hydrogen, or alkyl, haloalkyl, cycloalkyl, heterocycloalkyl, alkenyl, alkynyl, aryl, heteroaryl, aralkyl, or heteroaralkyl. In other embodiments, the phenyl is substituted with SR¹¹, —S(═O)OR¹¹, —S(═O)₂OR¹¹, —S(═O)₂N(R¹¹)₂, or —SC(═O)R¹¹. In another embodiment, the phenyl is substituted with SR¹¹, wherein R¹¹ may be hydrogen.

In certain embodiments, R is alkyl, heterocycloalkyl, alkenyl, alkynyl, aralkyl, or heteroaralkyl, wherein the alkyl, alkenyl, alkynyl, aralkyl, or heteroaralkyl may be optionally substituted with one or more groups selected from the group consisting of halo, alkyl, haloalkyl, aralkyl, alkenyl, alkynyl, cycloalkyl, heterocyclyl, aryl, heteroaryl, heteroaralkyl, hydroxy, alkoxy, aryloxy, heteroaryloxy, amino, nitro, sulfhydryl, amido, acyl, carboxyl, oxycarbonyl, acyloxy, thioether, sulfonate, sulfonyl, sulfonamido, formyl, cyano and isocyano. In other embodiments, R is alkyl, alkenyl or alkynyl. In other embodiments, R is a C₁ to C₁₀ alkyl. In other embodiments, R is n-pentyl, iso-pentyl, neo-pentyl or t-pentyl.

Another aspect of the invention relates to a substantially pure and isolated compound of formula II:

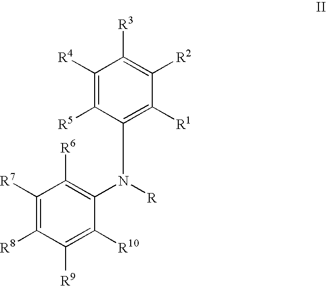

or a pharmaceutically acceptable salt thereof;
wherein, independently for each occurrence,
R is alkyl, alkenyl, alkynyl, aralkyl, or heteroaralkyl; and
R¹ to R¹⁰ are halo, azido, alkyl, aralkyl, alkenyl, alkynyl, cycloalkyl, heterocyclyl, aryl, heteroaryl, heteroaralkyl, hydroxy, alkoxy, aryloxy, heteroaryloxy, amino, alkylamino, arylamino, acylamino, heteroarylamino, nitro, sulfhydryl, imino, amido, phosphonate, phosphinate, acyl, carboxyl, oxycarbonyl, acyloxy, silyl, thioether, sulfonate, sulfonyl, sulfonamido, formyl, cyano or isocyano; wherein the aforementioned alkyl, alkenyl, alkynyl, cycloalkyl, heterocyclyl, aryl, heteroaryl, and heteroaralkyl may be optionally substituted with one or more groups selected from the group consisting halo, azido, alkyl, haloalkyl, fluoroalkyl, aralkyl, alkenyl, alkynyl, cycloalkyl, heterocyclyl, aryl, heteroaryl, heteroaralkyl, hydroxy, alkoxy, aryloxy, heteroaryloxy, amino, alkylamino, arylamino, acylamino, heteroarylamino, nitro, sulfhydryl, imino, amido, phosphonate, phosphinate, acyl, carboxyl, oxycarbonyl, acyloxy, silyl, thioether, sulfonate, sulfonyl, sulfonamido, formyl, cyano and isocyano.

In some embodiments, R is alkyl or alkenyl aralkyl or heteroalkyl. In other embodiments, R is C₁-C₆ alkyl. In still other embodiments, R is n-pentyl, isopentyl, neo-pentyl or t-pentyl.

In some embodiments, at least one of R¹, R², R³, R³ or R⁵ is halo, alkyl, haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹, —N(R¹¹)₂ or —N(R¹¹)C(═O)R¹¹; and R¹¹ is hydrogen, or alkyl, haloalkyl, cycloalkyl, heterocycloalkyl, alkenyl, alkynyl, aryl, heteroaryl, aralkyl, or heteroaralkyl. In other embodiments, at least one of R¹, R², R³, R³ or R⁵ is —N(R¹¹)₂ or —N(R¹¹)C(═O)R¹¹. In other embodiments, R¹, R², R³, R³ or R⁵ is —N(R¹¹)₂, wherein R¹¹ is hydrogen.

In some embodiments, at least one of R⁶, R⁷, R⁸, R⁹ or R¹⁰ is haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹, —N(R¹¹)C(═O)R¹¹; and R¹¹ is hydrogen, or alkyl, haloalkyl, cycloalkyl, heterocycloalkyl, alkenyl, alkynyl, aryl, heteroaryl, aralkyl, or heteroaralkyl. In other embodiments, at least one of R⁶, R⁷, R⁸, R⁹ or R¹⁰ is —SR¹¹, —S(═O)OR¹¹, —S(═O)₂OR¹¹, —S(═O)₂N(R¹¹)₂, or —SC(═O)R¹¹. In other embodiments, at least one of R⁶, R⁷, R⁸, R⁹ or R¹⁰ is —SR¹¹, wherein —R¹¹ is hydrogen.

In some embodiments, at least one of R¹, R², R⁴, R⁵, R⁶, R⁷, R⁹, and R¹⁰ is H.

Another aspect of the invention relates to a substantially pure and isolated compound represented by formula III:

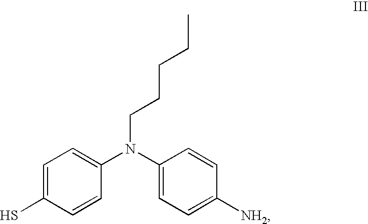

or a pharmaceutically acceptable salt thereof.

Another aspect of the invention relates to a pharmaceutical composition comprising any of the aforementioned compounds and a pharmaceutically acceptable carrier.

Another aspect of the method of treating or preventing a tryptase enzyme mediated condition in a subject in need thereof comprising administering to the subject an effective amount of the aforementioned compounds or pharmaceutical compositions. In some embodiments, the tryptase enzyme mediated condition is an inflammatory or allergic condition. In other embodiments, the tryptase enzyme mediated condition is allergic rhinitis, asthma, vascular injury, inflammatory bowel disease, psoriasis, arthritis, anaphylaxis, a wound, or an infection. In some embodiments, the vascular injury is restenosis or atherosclerosis. In some embodiments, the arthritis is rheumatoid arthritis, osteoarthritis or seronegative spondyloarthritis. The subject of the present invention may be a mammal. In some embodiments, the subject is a primate, such as a human.

Another aspect of the invention relates to a mixture comprising at least 10% of any of the aforementioned compounds. In some embodiments, the compound comprises at least 25%, or at least 75% of the mixture, or at least 95% of the mixture.

Another aspect of the invention relates to a compound of the present invention with tryptase inhibition activity in the range between 19 μM and 3.6 mM.

### BRIEF DESCRIPTION OF THE DRAWINGS

FIG. 1 depicts the dose-dependent inhibition of the tryptase enzyme with a compound of the present invention with an IC₅₀ of 493 μM (R²=0.98, n=27).

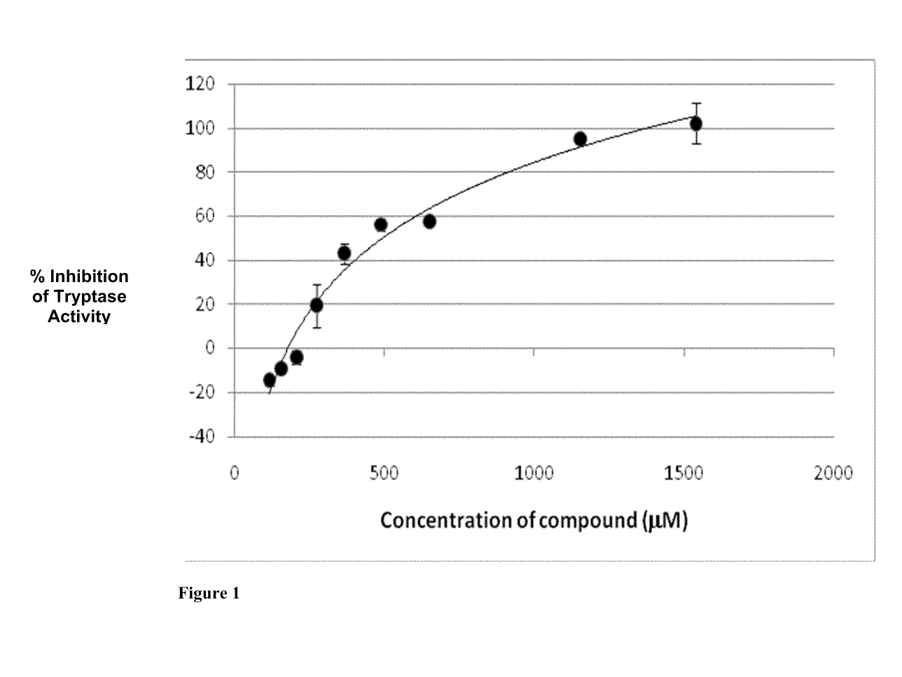

FIG. 2 depicts the interaction of a compound of the present invention with the tryptase enzyme active site indicating a strong hydrogen bond between the aromatic amine of compound III and Phe41 of the tryptase active site. In this orientation, the hydrocarbon “tail” of compound III is efficiently incorporated into the hydrophobic pocket of the active site created by the amino acid residues Val35, Val59, Gly60, and Leu64 increasing the stability of the bound inhibitor.

### DETAILED DESCRIPTION OF THE INVENTION

### Definitions

For convenience, before further description of the disclosure, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The term “acyl” as used herein refers to the radical

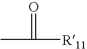

wherein R′₁₁ represents hydrogen, alkyl, alkenyl, alkynyl, or —(CH₂)ₘ—R₈₀, wherein R₈₀ is aryl, cycloalkyl, cycloalkenyl, heteroaryl or heterocyclyl; and m is an integer in the range 0 to 8, inclusive.

The term “alkyl” refers to a radical of a saturated straight or branched chain hydrocarbon group of, for example, 1-20 carbon atoms, or 1-12, 1-10, or 1-6 carbon atoms.

The term “alkenyl” refers to a radical of an unsaturated straight or branched chain hydrocarbon group of, for example, 2-20 carbon atoms, or 2-12, 2-10, or 2-6 carbon atoms, having at least one carbon-carbon double bond.

The term “alkynyl” refers to a radical of an unsaturated straight or branched chain hydrocarbon group of, for example, 2-20 carbon atoms, or 2-12, 2-10, or 2-6 carbon atoms, having at least one carbon-carbon triple bond.

The term “alkylene” or “alkylenyl” is art-recognized, and as used herein pertains to a bidentate moiety obtained by removing two hydrogen atoms from each of two different carbon atoms of a hydrocarbon compound. Examples of alkylene groups include, for example, —CH₂-(methylene), —CH₂CH₂— (ethylene), —CH₂CH₂CH₂— (propylene), —CH₂CH₂CH₂CH₂— (butylene), —CH₂CH₂CH₂CH₂CH₂— (pentylene), —CH₂CH₂CH₂CH₂CH₂CH₂— (hexylene), —CH(CH₃)—, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, —CH₂CH(CH₂CH₃)CH₂—, cyclopentylene (e.g., cyclopent-1,3-ylene), and cyclohexylene (e.g., cyclohex-1,4-ylene). As used herein “alkylene” includes substituted alkylene moieties (e.g. halogenated alkylenes).

The term “aliphatic” includes linear, branched, and cyclic alkanes, alkenes, or alkynes. In certain embodiments, aliphatic groups in the present invention are linear, branched or cyclic and have from 1 to about 20 carbon atoms.

The term “aralkyl” includes alkyl groups substituted with an aryl group or a heteroaryl group.

The term “heteroatom” includes an atom of any element other than carbon or hydrogen. Illustrative heteroatoms include boron, nitrogen, oxygen, phosphorus, sulfur and selenium, and alternatively oxygen, nitrogen or sulfur.

The term “halo” or “halogen” includes —F, —Cl, —Br, — or —I.

The term “perfluoro” refers to a hydrocarbon wherein all of the hydrogen atoms have been replaced with fluorine atoms. For example, —CF₃ is a perfluorinated methyl group.

The term “aryl” refers to a mono-, bi-, or other multi-carbocyclic, aromatic ring system. The aryl group can optionally be fused to one or more rings selected from aryls, cycloalkyls, and heterocyclyls. The aryl groups of this invention can be substituted with groups selected from alkyl, alkenyl, alkynyl, alkanoyl, alkoxy, alkylthio, amino, amido, aryl, aralkyl, azide, carbonyl, carboxy, cyano, cycloalkyl, ester, ether, halogen, haloalkyl, heterocyclyl, hydroxy, imino, ketone, nitro, perfluoroalkyl, phosphonate, phosphinate, silyl ether, sulfonamido, sulfonate, sulfonyl, and sulfhydryl.

The term “heteroaryl” refers to a mono-, bi-, or multi-cyclic, aromatic ring system containing one, two, or three heteroatoms such as nitrogen, oxygen, and sulfur. Examples include pyrrole, furan, thiophene, imidazole, oxazole, thiazole, triazole, pyrazole, pyridine, pyrazine, pyridazine and pyrimidine, and the like. Heteroaryls can also be fused to non-aromatic rings.

The terms “heterocycle”, “heterocyclyl”, or “heterocyclic” refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkyl, alkenyl, alkynyl, aldehyde, alkylthio, alkanoyl, alkoxy, alkoxycarbonyl, amido, amino, aminothiocarbonyl, aryl, arylcarbonyl, arylthio, carboxy, cyano, cycloalkyl, cycloalkylcarbonyl, ester, ether, halogen, heterocyclyl, heterocyclylcarbonyl, hydroxy, ketone, oxo, nitro, sulfonate, sulfonyl, and thiol.

Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl. Heterocycles also include bridged bicyclic groups where a monocyclic heterocyclic group can be bridged by an alkylene group.

The heterocyclic or heteroaryl ring may be and can be substituted with groups selected from alkyl, alkenyl, alkynyl, alkanoyl, alkoxy, alkoxy, alkylthio, amino, amido, aryl, aralkyl, azide, carbonyl, carboxy, cyano, cycloalkyl, ester, ether, halogen, haloalkyl, heterocyclyl, hydroxy, imino, ketone, nitro, perfluoroalkyl, phosphonate, phosphinate, silyl ether, sulfonamido, sulfonate, sulfonyl, and sulfhydryl.

The terms “polycyclyl” and “polycyclic group” include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above and can be substituted with groups selected from alkyl, alkenyl, alkynyl, alkanoyl, alkoxy, alkoxy, alkylthio, amino, amido, aryl, aralkyl, azide, carbonyl, carboxy, cyano, cycloalkyl, ester, ether, halogen, haloalkyl, heterocyclyl, hydroxy, imino, ketone, nitro, perfluoroalkyl, phosphonate, phosphinate, silyl ether, sulfonamido, sulfonate, sulfonyl, and sulfhydryl.

The term “carbocycle” includes an aromatic or non-aromatic ring in which each atom of the ring is carbon.

The terms “amine” and “amino” include both unsubstituted and substituted amines, e.g., a moiety that may be represented by the general formulas:

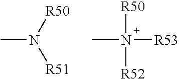

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

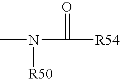

wherein R50 is as defined above, and R54 represents a hydrogen, an alkyl, an alkenyl or —(CH₂)ₘ—R61, where m and R61 are as defined above.

The term “amido” refers to an amino-substituted carbonyl and includes a moiety that may be represented by the general formula:

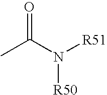

wherein R50 and R51 are as defined above. Certain embodiments of the amide in the present invention will not include imides that may be unstable.

The term “alkylthio” includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methyl thio, ethyl thio, and the like.

The term “carbonyl” includes such moieties as may be represented by the general formulas:

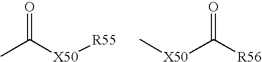

wherein X50 is a bond or represents an oxygen or a sulfur, and R55 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” includes a moiety that may be represented by the general formula:

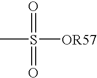

in which R57 is an electron pair, hydrogen, alkyl, cycloalkyl, or aryl.

The term “sulfate” includes a moiety that may be represented by the general formula:

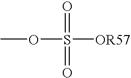

in which R57 is as defined above.

The term “sulfonamido” is art-recognized and includes a moiety that may be represented by the general formula:

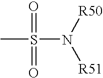

in which R50 and R51 are as defined above.

The term “sulfonyl” includes a moiety that may be represented by the general formula:

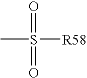

in which R58 is one of the following: hydrogen, alkyl, alkenyl, alkynyl, cycloalkyl, heterocyclyl, aryl or heteroaryl.

The term “sulfoxido” includes a moiety that may be represented by the general formula:

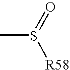

in which R58 is defined above.

As used herein, the term “substituted” is contemplated to include all permissible substituents of organic compounds. Illustrative substituents include, for example, those described herein above and as follows. Substitution may be by one or more groups such as alcohols, ethers, esters, amides, sulfones, sulfides, hydroxyl, nitro, cyano, carboxy, amines, heteroatoms, lower alkyl, lower alkoxy, lower alkoxycarbonyl, alkoxyalkoxy, acyloxy, halogen, trifluoromethoxy, trifluoromethyl, aralkyl, alkenyl, alkynyl, aryl, carboxyalkoxy, carboxyalkyl, cycloalkyl, cycloalkylalkyl, heterocyclyl, alkylheterocyclyl, heterocyclylalkyl, oxo, arylsulfonaminocarbonyl or any of the substituents of the preceding paragraphs or any of those substituents either attached directly or by suitable linkers. The linkers are typically short chains of 1-3 atoms containing any combination of —C—, —C(O)—, —NH—, —S—, —S(O)—, —O—, —C(O)O— or —S(O)—. For example, alkyl, alkenyl, alkynyl, aryl, cycloalkyl, heterocyclyl, acyl, amino, amido, etc. may be optionally substituted. In some embodiments, aforementioned groups may be optionally substituted with halogen, hydroxy, alkoxy, carboxy, carboxylic ester, nitro, cyano, amino, amido, alkyl, alkenyl, alkynyl, haloalkyl, cycloalkyl, aryl, heteroaryl, sulfonyl, or sulfonamido.

The term “optionally substituted” or “substituted” refers to a chemical group, such as alkyl, cycloalkyl, aryl, and the like, wherein one or more hydrogen atoms may be replaced with a substituent such as halogen, azide, alkyl, aralkyl, alkenyl, alkynyl, cycloalkyl, hydroxy, alkoxy, amino, amido, nitro, cyano, sulfhydryl, imino, phosphonate, phosphinate, carbonyl, carboxyl, silyl, ether, alkylthio, sulfonyl, sulfonamido, ketone, aldehyde, ester, heterocyclyl, aromatic or heteroaromatic moieties, perfluoroalkyl (e.g. —CF₃), acyl, and the like. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms.

The definition of each expression, e.g. alkyl, m, n, etc., when it occurs more than once in any structure, is intended to be independent of its definition elsewhere in the same structure unless otherwise indicated expressly or by the context.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

The term “hydrocarbon” includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2^{nd} ed., Wiley, New York, (1991).

The phrase “hydroxyl-protecting group” includes those groups intended to protect a hydroxyl group against undesirable reactions during synthetic procedures and includes, for example, benzyl or other suitable esters or ethers groups known in the art.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically active acid or base, followed by resolution of the diastereomers thus formed by fractional crystallization or chromatographic means well known in the art, and subsequent recovery of the pure enantiomers.

The term “effective amount” as used herein refers to the amount necessary to elicit the desired biological response. As will be appreciated by those of ordinary skill in this art, the effective amount of a drug may vary depending on such factors as the desired biological endpoint, the drug to be delivered, the composition of the encapsulating matrix, the target tissue, etc.

A “patient,” “subject” or “host” to be treated by the subject method may mean either a human or non-human animal.

As used herein, the term “tryptase” refers to the most abundant secretory granule-derived serine protease contained in mast cells that has recently been used as a marker for mast cell activation. It is involved with an allergenic response and is suspected to act as a mitogen for fibroblast lines.

As used herein, the term “inhibitor” refers to molecules that bind to enzymes and decrease their activity. The binding of an inhibitor can stop a substrate from entering the enzyme's active site and/or hinder the enzyme from catalyzing its reaction. Inhibitor binding is either reversible or irreversible. Irreversible inhibitors usually react with the enzyme and change it chemically. These inhibitors modify key amino acid residues needed for enzymatic activity. Reversible inhibitors bind non-covalently and different types of inhibition are produced depending on whether these inhibitors bind the enzyme, the enzyme-substrate complex, or both.

As used herein, the term “mast cell” refers to a resident cell of several types of tissues containing many granules rich in histamine and heparin. Although best known for their role in allergy and anaphylaxis, mast cells play an important protective role as well, being intimately involved in wound healing and defense against pathogens.

As used herein, the term “degranulation” refers to a cellular process that releases antimicrobial cytotoxic molecules from secretory vesicles called granules found inside some cells. It is used by several different cells involved in the immune system, including granulocytes (neutrophils, basophils and eosinophils) and mast cells, and certain lymphocytes such as natural killer (NK) cells and cytotoxic T cells, whose main purpose is to destroy invading microorganisms.

As used herein, the term “allergy” refers to a disorder of the immune system also referred to as atopy. Allergic reactions occur to environmental substances known as allergens; these reactions are acquired, predictable and rapid. Allergy is one of four forms of hypersensitivity and is called type I (or immediate) hypersensitivity. It is characterized by excessive activation of certain white blood cells called mast cells and basophils by a type of antibody known as IgE, resulting in an extreme inflammatory response. Common allergic reactions include eczema, hives, hay fever, asthma, food allergies, and reactions to the venom of stinging insects such as wasps and bees.

As used herein, the term “arthritis” refers to an inflammatory disorder that includes osteoarthritis and rheumatoid arthritis. The most common form of arthritis, osteoarthritis (degenerative joint disease) is a result of trauma to the joint, infection of the joint, or age. Other arthritis forms are rheumatoid arthritis and psoriatic arthritis, autoimmune diseases in which the body attacks itself. Septic arthritis is caused by joint infection. Gouty arthritis is caused by deposition of uric acid crystals in the joint, causing inflammation.

As used herein, the term “anaphylaxis” refers to an acute systemic (multi-system) and severe Type I Hypersensitivity allergic reaction in humans and other mammals causing anaphylactic shock due to the release of large quantities of immunological mediators (histamines, prostaglandins, leukotrienes) from mast cells leading to systemic vasodilation (associated with a sudden drop in blood pressure) and edema of bronchial mucosa (resulting in bronchoconstriction and difficulty breathing).

The compounds of the present invention may be used in the form of pharmaceutically acceptable salts derived from inorganic or organic acids. By “pharmaceutically acceptable salt” is meant those salts that are, within the scope of sound medical judgment, suitable for use in contact with the tissues of humans and lower animals without undue toxicity, irritation, and allergic response, and are commensurate with a reasonable benefit/risk ratio. Pharmaceutically acceptable salts are well known in the art. For example, S. M. Berge, et al. 1977, describe pharmaceutically-acceptable salts in J Pharm Sci. 66:1-19. The salts may be prepared in situ during the final isolation and purification of the compounds of the invention or separately by reacting a free base function with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates, such as dimethyl, diethyl, dibutyl and diamyl sulfates; long-chain halides such as decyl, lauryl, myristyl and stearyl chlorides, bromides and iodides; or arylalkyl halides, such as benzyl and phenethyl bromides and others. Water- or oil-soluble or -dispersible products are thereby obtained.

Examples of acids that may be employed to form pharmaceutically acceptable acid addition salts include such inorganic acids as hydrochloric acid, hydrobromic acid, sulfuric acid and phosphoric acid and such organic acids as oxalic acid, maleic acid, succinic acid, and citric acid.

The present invention includes all salts and all crystalline forms of such salts. Basic addition salts can be prepared in situ during the final isolation and purification of compounds of this invention by combining a carboxylic acid-containing group with a suitable base such as the hydroxide, carbonate, or bicarbonate of a pharmaceutically acceptable metal cation or with ammonia or an organic primary, secondary, or tertiary amine. Pharmaceutically acceptable basic addition salts include cations based on alkali metals or alkaline earth metals such as lithium, sodium, potassium, calcium, magnesium, and aluminum salts, and nontoxic quaternary ammonia and amine cations including ammonium, tetramethylammonium, tetraethylammonium, methylamine, dimethylamine, trimethylamine, triethylamine, diethylamine, and ethylamine. Other representative organic amines useful for the formation of base addition salts include ethylenediamine, ethanolamine, diethanolamine, piperidine, and piperazine.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “preventing”, when used in relation to a condition, such as cancer, an infectious disease, or other medical disease or condition, is well understood in the art, and includes administration of a composition which reduces the frequency of, or delays the onset of, symptoms of a medical condition in a subject relative to a subject which does not receive the composition. Thus, prevention of an infection includes, for example, reducing the number of diagnoses of the infection in a treated population versus an untreated control population, and/or delaying the onset of symptoms of the infection in a treated population versus an untreated control population.

The term “synergistic” refers to two or more components working together so that the total effect is greater than the sum of the components.

The term “treating” is art-recognized and refers to curing as well as ameliorating at least one symptom of any condition or disorder.

### Compounds

The present invention relates to novel compounds and pharmaceutical compositions comprising these compounds. In one aspect, the invention relates to a substantially pure and isolated compound of formula I:

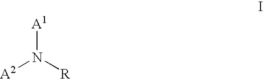

or a pharmaceutically acceptable salt thereof,
wherein, independently for each occurrence,
A¹ and A² are each aryl; and
R is alkyl, cycloalkyl, heterocycloalkyl, alkenyl, alkynyl, aryl, heteroaryl, aralkyl, or heteroaralkyl;
wherein any of the aforementioned alkyl, cycloalkyl, heterocycloalkyl, alkenyl, alkynyl, aryl, heteroaryl, aralkyl, or heteroaralkyl may be optionally substituted with one or more groups selected from the group consisting of halo, azido, alkyl, haloalkyl, aralkyl, alkenyl, alkynyl, cycloalkyl, heterocyclyl, aryl, heteroaryl, heteroaralkyl, hydroxy, alkoxy, aryloxy, heteroaryloxy, amino, nitro, sulfhydryl, imino, amido, phosphonate, phosphinate, acyl, carboxyl, oxycarbonyl, acyloxy, silyl, thioether, sulfonate, sulfonyl, sulfonamido, formyl, cyano and isocyano.

In some embodiments, A¹ and A² are phenyl or napthyl. In other embodiments, at least one of A¹ and A² is phenyl, while in other embodiments, both A¹ and A² are phenyl. In some embodiments, A¹ is a phenyl, which may be substituted with at least one of a halo, alkyl, haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹, —N(R¹¹)₂ or —N(R¹¹)C(═O)R¹¹; and R¹¹ is hydrogen, or alkyl, haloalkyl, cycloalkyl, heterocycloalkyl, alkenyl, alkynyl, aryl, heteroaryl, aralkyl, or heteroaralkyl. In other embodiments, the phenyl is substituted with —N(R¹¹)₂ or —N(R¹¹)C(═O)R¹¹. In another embodiment, the phenyl is substituted with —N(R¹¹)₂, wherein R¹¹ is hydrogen, alkyl, or aralkyl. In other embodiments, R¹¹ is hydrogen, methyl, ethyl, propyl or isopropyl.

In some embodiments, A² is phenyl, which may be substituted with at least one of a halo, alkyl, haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —(═O)R¹¹; —N(R¹¹)₂ or —N(R¹¹)C(═O)R¹¹; and R¹¹ is hydrogen, or alkyl, haloalkyl, cycloalkyl, heterocycloalkyl, alkenyl, alkynyl, aryl, heteroaryl, aralkyl, or heteroaralkyl. In other embodiments, the phenyl is substituted with SR¹¹, —S(═O)OR¹¹, —S(═O)₂OR¹¹, —S(═O)₂N(R¹¹)₂, or —SC(═O)R¹¹. In other embodiments, the phenyl is substituted with SR¹¹, wherein R¹¹ may be hydrogen, methyl, ethyl, propyl or isopropyl. In other embodiments, R¹¹ is hydrogen.

In certain embodiments, R is alkyl, heterocycloalkyl, alkenyl, alkynyl, aralkyl, or heteroaralkyl, each of which may be optionally substituted with one or more groups selected from the group consisting of halo, alkyl, haloalkyl, aralkyl, alkenyl, alkynyl, cycloalkyl, heterocyclyl, aryl, heteroaryl, heteroaralkyl, hydroxy, alkoxy, aryloxy, heteroaryloxy, amino, nitro, sulfhydryl, amido, acyl, carboxyl, oxycarbonyl, acyloxy, thioether, sulfonate, sulfonyl, sulfonamido, formyl, cyano and isocyano. In other embodiments, R is alkyl, alkenyl or alkynyl. In other embodiments, R is a C₁ to C₁₀ alkyl. In other embodiments, R is n-pentyl, iso-pentyl, neo-pentyl or t-pentyl.

Another aspect of the invention relates to a substantially pure and isolated compound of formula II:

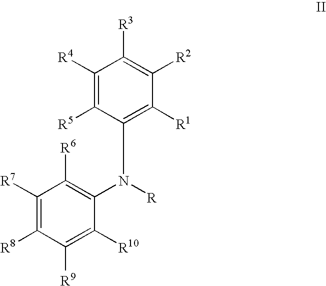

or a pharmaceutically acceptable salt thereof;
wherein, independently for each occurrence,
R is alkyl, alkenyl, alkynyl, aralkyl, or heteroaralkyl; and
R¹ to R¹⁰ are halo, azido, alkyl, aralkyl, alkenyl, alkynyl, cycloalkyl, heterocyclyl, aryl, heteroaryl, heteroaralkyl, hydroxy, alkoxy, aryloxy, heteroaryloxy, amino, alkylamino, arylamino, acylamino, heteroarylamino, nitro, sulfhydryl, imino, amido, phosphonate, phosphinate, acyl, carboxyl, oxycarbonyl, acyloxy, silyl, thioether, sulfonate, sulfonyl, sulfonamido, formyl, cyano or isocyano; wherein the aforementioned alkyl, alkenyl, alkynyl, cycloalkyl, heterocyclyl, aryl, heteroaryl, and heteroaralkyl may be optionally substituted with one or more groups selected from the group consisting halo, azido, alkyl, haloalkyl, fluoroalkyl, aralkyl, alkenyl, alkynyl, cycloalkyl, heterocyclyl, aryl, heteroaryl, heteroaralkyl, hydroxy, alkoxy, aryloxy, heteroaryloxy, amino, alkylamino, arylamino, acylamino, heteroarylamino, nitro, sulfhydryl, imino, amido, phosphonate, phosphinate, acyl, carboxyl, oxycarbonyl, acyloxy, silyl, thioether, sulfonate, sulfonyl, sulfonamido, formyl, cyano and isocyano.

In some embodiments, R is alkyl or alkenyl aralkyl or heteroalkyl. In other embodiments, R is C₁-C₆ alkyl. In still other embodiments, R is n-pentyl, isopentyl, neo-pentyl or t-pentyl.

In some embodiments, at least one of R¹, R², R³, R³ or R⁵ is halo, alkyl, haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹—N(R¹¹)₂ or —N(R¹¹)C(═O)R¹¹; and R¹¹ is hydrogen, or alkyl, haloalkyl, cycloalkyl, heterocycloalkyl, alkenyl, alkynyl, aryl, heteroaryl, aralkyl, or heteroaralkyl. In other embodiments, at least one of R¹, R², R³, R³ or R⁵ is —N(R¹¹)₂ or —N(R¹¹)C(═O)R¹¹. In other embodiments, R¹, R², R³, R³ or R⁵ is —N(R¹¹)₂, wherein R¹¹ is hydrogen. In other embodiments, one of R¹, R², R³, R³ or R⁵ is halo, alkyl, haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹, —N(R¹¹)₂ or —N(R¹¹)C(═O)R¹¹; and the remaining four of R¹, R², R³, R³ or R⁵ are each hydrogen.

In some embodiments, at least one of R⁶, R⁷, R⁸, R⁹ or R¹⁰ is haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹, —N(R¹¹)₂ or —N(R¹¹)C(═O)R¹¹; and R¹¹ is hydrogen, or alkyl, haloalkyl, cycloalkyl, heterocycloalkyl, alkenyl, alkynyl, aryl, heteroaryl, aralkyl, or heteroaralkyl. In other embodiments, at least one of R⁶, R⁷, R⁸, R⁹ or R¹⁰ is —SR¹¹, —S(═O)OR¹¹, —S(═O)₂OR¹¹, —S(═O)₂N(R¹¹)₂, or —SC(═O)R¹¹. In other embodiments, at least one of R⁶, R⁷, R⁸, R⁹ or R¹⁰ is —SR¹¹, wherein —R¹¹ is hydrogen. In some embodiments, one of R⁶, R⁷, R⁸, R⁹ and R¹⁰ is haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹, —N(R¹¹)₂ or —N(R¹¹)C(═O)R¹¹, and the remaining four of R⁶, R⁷, R⁸, R⁹ or R¹⁰ are each hydrogen.

In some embodiments, at least one of R¹, R², R⁴, R⁵, R⁶, R⁷, R⁹, and R¹⁰ is H.

Another aspect of the invention relates to a substantially pure and isolated compound represented by formula III:

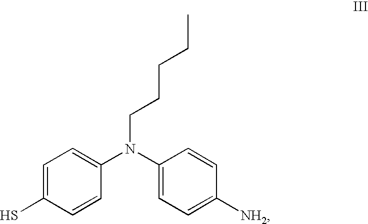

or a pharmaceutically acceptable salt thereof.

Another aspect of the invention relates to a mixture comprising at least 10% of any of the aforementioned compounds. In some embodiments, compound comprises at least 25% of the mixture, while in other embodiments, the compounds comprises at least 75% or at least 95% of the mixture.

Another aspect of the invention relates to a compound of the present invention with tryptase inhibition ranging between 19 μM and 3.6 mM.

### Synthesis of Compounds of the Invention

Scheme I shows a general scheme for preparing compounds of formula I. Combining an anline compound (1) with an aryl chloride (2) in the solvent DMF and potassium carbonate base provides a diaryl amine (3). This amine can be further alkylated by a reaction with an R—X compound, wherein X is a leaving group, such as a bromine, to provide a compound of formula I. The aryl and R groups may be further functionalized using methods known in the art.

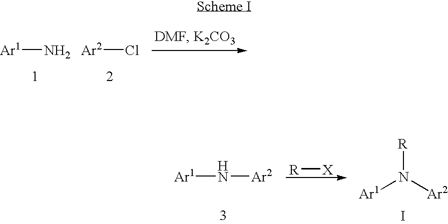

Scheme II shows the synthesis of a compound of the present invention (III). Briefly, 4-aminothiphenol (4) and 4-chloronitrobenzene (5) were combined with potassium carbonate in DMF to provide the nitrothio amine compound 6. NaH and DMF were used to facilitate the addition of bromopentane to the central secondary amine giving compound 7. Reduction of the para-nitro group to the aniline was accomplished using tin chloride (SnCl₂) yielding compound 3 as a free base. The HCl salt was prepared by adding concentrated HCl to the free base of compound III in ether. The resulting salt precipitates out and can be collected through filtration.

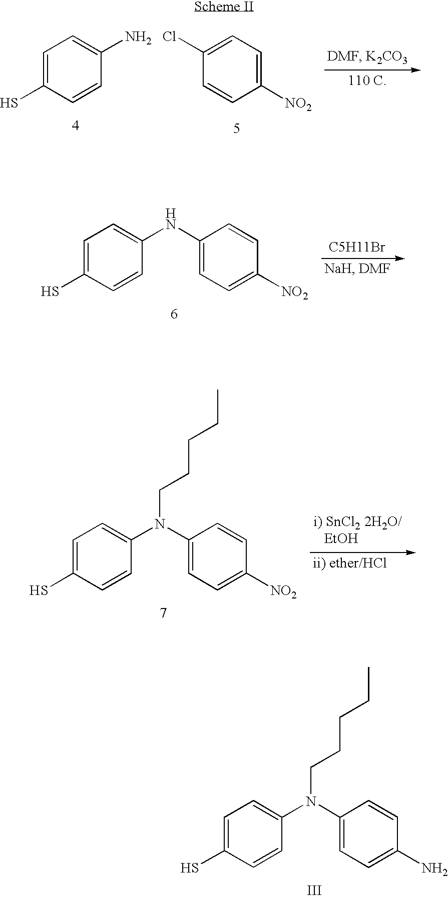

### Pharmaceutical Compositions

Another aspect of the invention provides pharmaceutical compositions comprising the aforementioned compounds formulated together with one or more pharmaceutically acceptable carriers. The pharmaceutical compositions may be formulated specially for topical administration. Alternatively, the pharmaceutical compositions may be formulated specially for oral administration in solid or liquid form, for parenteral injection, for rectal administration, or for vaginal administration. The pharmaceutical compositions may encompass crystalline and amorphous forms of the active ingredient(s).

As used herein, the phrase “pharmaceutically acceptable carrier” refers to any and all solvents, dispersion media, coatings, antibacterial and antifungal agents, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

The pharmaceutical compositions can be administered to humans and other animals orally, rectally, parenterally, intracisternally, intravaginally, intraperitoneally, topically (as by powders, ointments, or drops), buccally, or as an oral or nasal spray. The compositions may also be administered through the lungs by inhalation. The term “parenteral administration” as used herein refers to modes of administration, which include intravenous, intramuscular, intraperitoneal, intracisternal, subcutaneous and intra-articular injection and infusion.

Pharmaceutical compositions for parenteral injection comprise pharmaceutically-acceptable aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, and polyethylene glycol), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservatives, wetting agents, emulsifying agents, and dispersing agents. They may also contain taggants or other anti-counterfeiting agents, which are well known in the art. Prevention of the action of microorganisms may be ensured by the inclusion of various antibacterial and antifungal agents, for example, paraben, chlorobutanol, and phenol sorbic acid. It may also be desirable to include isotonic agents such as sugars, and sodium chloride. Prolonged absorption of the injectable pharmaceutical form may be brought about by the inclusion of agents, which delay absorption such as aluminum monostearate and gelatin.

In some cases, in order to prolong the effect of the drug, it may be desirable to slow the absorption of the drug following subcutaneous or intramuscular injection. This may be accomplished by the use of a liquid suspension of crystalline or amorphous material with poor water solubility. Amorphous material may be used alone or together with stabilizers as necessary. The rate of absorption of the drug then depends upon its rate of dissolution, which in turn, may depend upon crystal size and crystalline form.

Alternatively, delayed absorption of a parenterally administered drug form can be accomplished by dissolving or suspending the drug in an oil vehicle.

Injectable depot forms can be made by forming microencapsulating matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations can also be prepared by entrapping the drug in liposomes or microemulsions, which are compatible with body tissues.

The injectable formulations can be sterilized, for example, by filtration through a bacterial-retaining filter, or by incorporating sterilizing agents in the form of sterile solid compositions, which can be dissolved or dispersed in sterile water or other sterile injectable medium just prior to use.

Solid dosage forms for oral administration include capsules, tablets, pills, powders, and granules. Such forms may include forms that dissolve or disintegrate quickly in the oral environment. In such solid dosage forms, the active compound can be mixed with at least one inert, pharmaceutically acceptable excipient or carrier. Suitable excipients include, for example, (a) fillers or extenders such as starches, lactose, sucrose, glucose, mannitol, and silicic acid; (b) binders such as cellulose and cellulose derivatives (such as hydroxypropylmethylcellulose, hydroxypropylcellulose, and carboxymethylcellulose), alginates, gelatin, polyvinylpyrrolidone, sucrose, and acacia; (c) humectants such as glycerol; (d) disintegrating agents such as sodium starch glycolate, croscarmellose, agar-agar, calcium carbonate, potato or tapioca starch, alginic acid, certain silicates, and sodium carbonate; (e) solution retarding agents such as paraffin; (f) absorption accelerators such as quaternary ammonium compounds; (g) wetting agents, such as cetyl alcohol and glycerol monostearate, fatty acid esters of sorbitan, poloxamers, and polyethyleneglycols; (h) absorbents such as kaolin and bentonite clay; (i) lubricants such as talc, calcium stearate, magnesium stearate, solid polyethylene glycols, sodium lauryl sulfate, and mixtures thereof; and (j) glidants such as talc, and silicone dioxide. Other suitable excipients include, for example, sodium citrate or dicalcium phosphate. The dosage forms may also comprise buffering agents.

Solid or semi-solid compositions of a similar type may also be employed as fillers in soft and hard-filled gelatin capsules using such excipients as lactose or milk sugar as well as high molecular weight polyethylene glycols.

Solid dosage forms, including those of tablets, dragees, capsules, pills, and granules, can be prepared with coatings and shells such as functional and aesthetic enteric coatings and other coatings well known in the pharmaceutical formulating art. They may optionally contain opacifying agents and colorants. They may also be in a form capable of controlled or sustained release. Examples of embedding compositions that can be used for such purposes include polymeric substances and waxes.

The active compounds can also be in micro-encapsulated form, if appropriate, with one or more of the above-mentioned excipients.

Liquid dosage forms include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers such as cyclodextrins, ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, and perfuming agents. Other ingredients include flavorants for dissolving or disintegrating oral or buccal forms.

Suspensions, in addition to the active compounds, may contain suspending agents such as, for example, ethoxylated isostearyl alcohols, polyoxyethylene sorbitol and sorbitan esters, cellulose or cellulose derivatives (for example microcrystalline cellulose), aluminum metahydroxide, bentonite, agar agar, and tragacanth, and mixtures thereof.

Compositions for rectal or vaginal administration may be suppositories that can be prepared by mixing the compounds of this invention with suitable nonirritating excipients or carriers such as cocoa butter, polyethylene glycol or a suppository wax, that are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes can be formed by lipid monolayer, bilayer, or other lamellar or multilamellar systems that are dispersed in an aqueous medium. Any nontoxic, physiologically-acceptable and metabolizable lipid capable of forming liposomes can be used. The present compositions in liposome form can contain, in addition to a compound of the present invention, stabilizers, preservatives, and excipients. Exemplary lipids include the phospholipids and the phosphatidyl cholines (lecithins), both natural and synthetic. Methods to form liposomes are known in the art. See, for example, Prescott, Ed., Methods in Cell Biology, Volume XIV, Academic Press, New York (1976), p. 33 et seq.

A buffer may be beneficial in specific formulations. Preferred buffering agents include mono- and di-sodium phosphates and borates, basic magnesium carbonate and combinations of magnesium and aluminum hydroxide.

In one implementation, the tableting powder is made by mixing in a dry powdered form the various components as described above, e.g., active ingredient (curcuma species extract composition), diluent, sweetening additive, and flavoring, etc. An average in the range of about 10% to about 15% by weight of the active extract of the active ingredient can be added to compensate for losses during subsequent tablet processing. The mixture is then sifted through a sieve with a mesh size preferably in the range of about 80 mesh to about 100 mesh to ensure a generally uniform composition of particles. The tablet can be of any desired size, shape, weight, or consistency.

### Delivery Systems

Administration modes useful for the delivery of the compositions of the present invention to a subject include administration modes commonly known to one of ordinary skill in the art, such as, for example, powders, sprays, ointments, pastes, creams, lotions, gels, solutions, patches and inhalants.

In one embodiment, the delivery system may be an inhalation delivery system, such as, for example, an inhaler or nebulizer.

In another embodiment, the delivery system may be a transdermal delivery system, such as, for example, a hydrogel, cream, lotion, ointment, or patch. A patch in particular may be used when a timed delivery of weeks or even months is desired.

In another embodiment, parenteral routes of administration may be used. Parenteral routes involve injections into various compartments of the body. Parenteral routes include intravenous (iv), i.e. administration directly into the vascular system through a vein; intra-arterial (ia), i.e. administration directly into the vascular system through an artery; intraperitoneal (ip), i.e. administration into the abdominal cavity; subcutaneous (sc), i.e. administration under the skin; intramuscular (im), i.e. administration into a muscle; and intradermal (id), i.e. administration between layers of skin. The parenteral route is sometimes preferred over oral ones when part of the formulation administered would partially or totally degrade in the gastrointestinal tract. Similarly, where there is need for rapid response in emergency cases, parenteral administration is usually preferred over oral.

### Methods of Treatment

Methods of the present invention comprise providing the aforementioned compounds for the treatment and/or prevention of diseases and disorders involving the tryptase enzyme. For example, the composition of the present invention may be useful for treating or preventing allergic rhinitis, asthma, vascular injury (e.g., restenosis and atherosclerosis), inflammatory bowel disease, psoriasis, arthritis, anaphylaxis, wounds, infections, and other allergy and inflammatory related diseases in a mammal, such as a human.

The foregoing description includes the best presently contemplated mode of carrying out the present invention. This description is made for the purpose of illustrating the general principles of the inventions and should not be taken in a limiting sense. This invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

### Exemplification

Compound [III], as the free base, was identified from a botanical as described below.

### Methods

A. Tryptase Enzyme Inhibition

Tryptase activity was determined by monitoring the production of chromophore p-nitroaniline (pNA) generated by the cleavage of tosyl-gly-pro-lys-pNA by the tryptase enzyme according to the manufacturer's protocol (Millipore Inc., Westbury, Mass.). In a 96-well format, 10 μL of tryptase was added to 10 μL of sample, followed by 20 μL of tosyl-gly-pro-lys-pNA and 160 μL of 1× reaction buffer and incubated for 2 h at 37° C. After the incubation, absorbance at 405 nm was measured in each well using a Tecan M200 microplate reader.

B. DART Time-of-Flight Mass Spectrometry

The JEOL DART™ AccuTOF mass spectrometer (JMS-T 100LC; Jeol USA, Peabody, Mass.) used for chemical analysis requires no sample preparation and yields masses with accuracies to 0.0001 mass units (R. B. Cody, J. A. Larameé, J. M. Nilles, and H. D. Durst, 2005. Direct Analysis in Real Time (DART™) Mass Spectrometry. JEOL News 40:8-12). For positive ion mode (DART+), the needle voltage was set to 3000V, heating element to 250° C., electrode 1 to 150V, electrode 2 to 250V, and helium gas flow to 2.52 liters per min. For the mass spectrometer, the following settings were loaded: orifice 1 set to 10V, ring lens voltage set to 5V, and orifice 2 set to 5V. The peak voltage was set to 1000V in order to give peak resolution beginning at 100 m/z. The microchannel plate detector (MCP) voltage was set at 2600V. Calibrations were performed internally with each sample using a 10% (w/v) solution of PEG that provided mass markers throughout the required mass range 100-1000 m/z. Calibration tolerances were held to 5 mmu.

C. Determination of Chemical Structures

Molecular formula and chemical structure was identified and confirmed by elemental composition and isotope matching programs in the Jeol MassCenterMain Suite software (MassCenter Main, Version 1.3.0.0; JEOL USA Inc.: Peabody, Mass., USA, Copyright® 2001-2004). In addition, molecular formulas and structure identifications were searched against the NIST/NIH/EPA Mass Spec Database (S. Stein, Y. Mirokhin, D. Tchekhovskoi, G. Mallard, A. Mikaia, V. Zaikin, J. Little, D. Zhu, C. Clifton, and D. Sparkman, 2005. The NIST mass spectral search program for the NIST/EPA/NIH mass spectral library—Version 2.0d. National Institute of Standards and Technology, Gaithersburg, Md.), the Dictionary of Natural Products (Chapman & Hall: Dictionary of Natural Products on DVD Version 16:2. CRC Press, Boca Raton, Fla., 2008), and the Chemical Abstract Services structure search (chembiofinder.cambridgesoft.com).

D. Pharmacokinetic Analysis

Five healthy consenting female adults ranging in age from 23 to 57 were took diets free of flavonoids and any NSAIDs. A certified individual collected blood samples at several time intervals between 0 and 480 min after compounds of the present invention were ingested in a mixture. Immediately after the time zero blood samples were collected, a single 100 mg dose of the composition was administered as a lozenge. Blood samples were handled with approved protocols and precautions, centrifuged to remove cells and the serum fraction was collected and frozen. Blood was not treated with heparin to avoid any analytical interference. Urine samples were collected from the same five subjects on a time course (0 to 8 h).

The cells were removed from the blood samples by centrifugation and the serum was collected. Serum samples were prepared for DART TOF-MS analysis by extraction with an equal volume of neat ethanol (USP) to minimize background of proteins, peptides, and polysaccharides present in serum. The ethanol extract was centrifuged for 10 min at 4° C., the supernatant was removed, concentrated to 200 μL volume, and 50 μL of an internal standard was added. Urine samples were not treated and used directly for DART TOF-MS. DART TOF-MS analyses were conducted as described above.

### Results

A. Identification of Compounds of the Present Invention

Through the use of DART fingerprinting as well as a proprietary method for identifying in vitro bioactive chemicals in botanical extracts, it was possible to determine which chemicals were inhibiting tryptase activity in a mixture of compounds. The chemical structures of the tryptase inhibitors were determined based upon isotopic ratio matching of the determined molecular formulas from the DART AccuTOF-MS analysis as well as molecular modeling. The identified aminopyridine tryptase inhibitor was identified at m/z (M+H')=287.1582 and possessed a molecular formula of C₁₇H₂₂N₂S.

B. Tryptase Inhibition

The IC₅₀ values for tryptase inhibition ranged between 19 μM and 3.6 mM for compounds of the present invention. Synthesized compound III as the HCl salt (Section E below) inhibits tryptase activity with an IC₅₀ value of 493 μM relative to controls.

C. Absorption, Distribution, Metabolism, Excretion, and Toxicity (ADMET) Predictions

Molecular modeling software was used to predict the Absorption, Distribution, Metabolism, Excretion, and Toxicity (ADMET) properties of the pharmaceutical compositions of the present invention. The physicochemical properties of the compounds of the present invention were used for the ADMET evaluations. Based on the calculations, compounds of the present invention will be absorbed in the small intestine, are likely to pass through the blood brain barrier, and are not likely to be hepatotoxic. Using similar molecular modeling tools, it was determined that compounds of the present invention are not mutagenic, based on AMES mutagenicity predictions. The acute oral toxicity was estimated to be 900 mg Kg⁻¹.

D. Pharmacokinetic Properties

The compounds of the present invention, particularly compound [III], when present in a mixture and ingested by humans in the form of a slow-dissolve lozenge is found in the bloodstream (serum) within 10 min. Compound [III] is present in the blood up to 360 min post-ingestion and was not detected at 480 min (6 h) after ingestion. The very rapid uptake of compound [III] suggests oral cavity uptake. Compound [III] appears in urine within 1 h and is present in urine up to 8 h post-ingestion.

E. Molecular Modeling

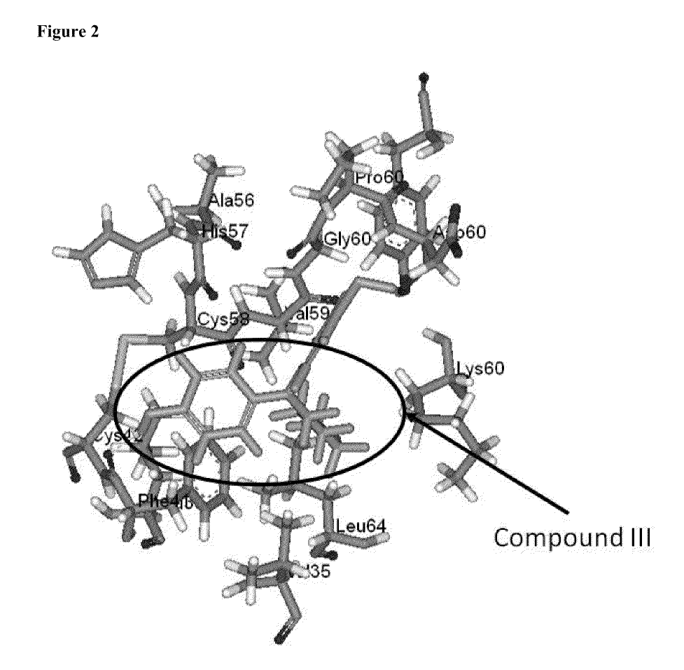

While not being bound by any particular theory, it is believed that the compounds of the present invention as exemplified by compound [III] enters the hydrophobic pocket of the tryptase active site created by the amino acid residues Val35, Val59, Gly60, and Leu64. The hydrophobic active site will stabilize compounds of the present invention that contain hydrocarbon and other hydrophobic functional groups. Further stabilization of compound [III] and other compounds of the present invention containing aromatic hydrogen donors including, but not limited to alcohol, amine, and thiol groups, will occur through hydrogen bonding with Phe41 at the entrance to the active site. When bound, compounds of the present invention are efficiently incorporated into the tryptase active site, thereby inhibiting the proteolytic activity of the tryptase enzyme (See FIG. 2).

F. Synthesis of a Compound of the Present Invention

Preparation of 4-(4-Nitro-phenylamino)-benzenethiol [10]: 4-Amino thiophenol ([8], 1.0 g, 8.0 mmol), 4-chloronitro benzene ([9], 1.3 g, 8.25 mmol), potassium carbonate (2.8 g, 20 mmol) and dry N,N′-dimethylformamide (30 mL) were combined in a dry flask, heated to 110° C. and maintained at this temperature for 12 h. After cooling, the reaction mixture was poured into ice cold water (100 mL) and extracted with ethyl acetate (2×75 mL). The combined organic layer was washed with water (2×150 mL) followed by brine (15%, 150 mL) and dried over sodium sulfate. The filtered organic layer was concentrated under vacuum to give a dark solid which was purified by silica gel column chromatography using hexanes and ethyl acetate (95:5, 500 mL), followed by hexanes:ethyl acetate (85:15, 500 mL). The fractions collected from the 85:15 elution were combined and concentrated under vacuum to give [10] as an orange yellow solid (1.6 g, yield: 81%).

4-[(4-Nitro-phenyl)-pentyl-amino]benzenethiol [11]: In a 100 mL 3-necked RB flask, NaH (60% in mineral oil, 0.234 g, 9.75 mmol) and dry DMF were mixed and cooled to 5° C. A solution of compound [10] (2.0 g, 8.13 mmol) in dry DMF was added slowly over a period of 20 min while maintaining the temperature of the reaction below 5° C. The reaction mixture was allowed to warm to room temperature and maintained for 60 min. The reaction mixture was again cooled to 5° C. and bromopentane (1.47 g, 9.73 mmol) was added slowly over a period of 30 min. The reaction mixture was slowly heated to 50° C. and maintained for 12 h. The cooled reaction mixture was poured into ice-cold water (100 mL) and extracted with ethyl acetate (2×100 mL). The combined organic layer was washed with water (2×150 mL) followed by brine (15%, 150 mL) and dried over sodium sulfate (˜100 g). The filtered organic layer was concentrated under vacuum to give a dark sold which was purified by silica gel column chromatography eluted with hexanes and ethyl acetate (97:3, 200 mL) followed by hexanes and ethyl acetate (90:10, 300 mL)]. The 90:10 fractions were combined and concentrated under vacuum to give [11] as a yellow solid (1.82 g, yield: 71%).

4-[(4-Amino-phenyl)-pentyl-amino]benzenethiol hydrochloride salt [III]: Compound ([11], 2.28 gm, 7.2 mmol) was added to a mixture of ethanol (30 mL), conc. HCl (0.5 mL) and tin (II) chloride.dihydrate (6.5 gm, 28 mmol) and refluxed for 60 min. The reaction mixture was cooled to room temperature and concentrated under vacuum. The resulting residue was dissolved in water (50 mL) and the pH was adjusted to 7.5 with saturated sodium bicarbonate. The aqueous layer was extracted with hot (˜50° C.) ethyl acetate (3×50 mL) and the organic layer was washed with water (75 mL) and brine (75 mL), dried over sodium sulfate, filtered, and concentrated to give the crude compound (free base [III], ˜3.0 g) which was purified by silica gel column chromatography eluted with 200 mL hexanes:ethyl acetate (85:15), followed by 300 mL hexanes:ethyl acetate (80:20), and finally followed by 300 mL hexanes:ethyl acetate (75:25). These final fractions (75:25; hexanes:ethyl acetate) were collected, combined, and concentrated under vacuum to give the free base of [III] as pale yellow oil (1.64 g, yield: 80%).

The free base of [III] was dissolved in diethyl ether (50 mL) and cooled to 10° C. Then 1 M HCl in ether (11.5 mL, 2.0 eq) was added slowly over a period of 15 min and stirred for 60 min at 25-30° C. The precipitated solid was filtered (under nitrogen atmosphere), washed with ether, immediately transferred into a flask and high vacuum was applied to give compound [III] as an off-white powder (weight: 0.96 g, yield: 52%).

1. A substantially pure and isolated compound of formula I: 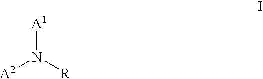
or a pharmaceutically acceptable salt thereof,
wherein, independently for each occurrence,
A¹ and A² are each aryl; and
R is alkyl, cycloalkyl, heterocycloalkyl, alkenyl, alkynyl, aryl, heteroaryl, aralkyl, or heteroaralkyl;
wherein any of the aforementioned alkyl, cycloalkyl, heterocycloalkyl, alkenyl, alkynyl, aryl, heteroaryl, aralkyl, or heteroaralkyl may be optionally substituted with one or more groups selected from the group consisting of halo, azido, alkyl, haloalkyl, aralkyl, alkenyl, alkynyl, cycloalkyl, heterocyclyl, aryl, heteroaryl, heteroaralkyl, hydroxy, alkoxy, aryloxy, heteroaryloxy, amino, nitro, sulfhydryl, imino, amido, phosphonate, phosphinate, acyl, carboxyl, oxycarbonyl, acyloxy, silyl, thioether, sulfonate, sulfonyl, sulfonamido, formyl, cyano and isocyano.

2. The compound of claim 1, wherein A¹ is a phenyl.

3. The compound of claim 2, wherein the phenyl is substituted with at least one of a halo, alkyl, haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹, —N(R¹¹)₂ or —N(R¹¹)C(═O)R¹¹; and R¹¹ is hydrogen, or alkyl, haloalkyl, cycloalkyl, heterocycloalkyl, alkenyl, alkynyl, aryl, heteroaryl, aralkyl, or heteroaralkyl.

4. The compound of claim 3, wherein the phenyl is substituted with —N(R¹¹)₂ or —N(R¹¹)C(═O)R¹¹.

5. The compound of claim 4, wherein the phenyl is substituted with —N(R¹¹)₂.

6. The compound of claim 5, wherein R¹¹ is hydrogen.

7. The compound of claim 1, wherein A² is phenyl.

8. The compound of claim 7, wherein the phenyl is substituted with at least one of a halo, alkyl, haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹, —N(R¹¹)₂ or —N(R¹¹)C(═O)R¹¹; and R¹¹ is hydrogen, or alkyl, haloalkyl, cycloalkyl, heterocycloalkyl, alkenyl, alkynyl, aryl, heteroaryl, aralkyl, or heteroaralkyl.

9. The compound of claim 8, wherein the phenyl is substituted with SR¹¹, —S(═O)OR¹¹, —S(═O)₂OR¹¹, —S(═O)₂N(R¹¹)₂, or —SC(═O)R¹¹.

10. The compound of claim 9, wherein the phenyl is substituted with SR¹¹.

11. The compound of claim 10, wherein R¹¹ is hydrogen.

12. The compound of claim 1, wherein R is alkyl, heterocycloalkyl, alkenyl, alkynyl, aralkyl, or heteroaralkyl, wherein the alkyl, alkenyl, alkynyl, aralkyl, or heteroaralkyl may be optionally substituted with one or more groups selected from the group consisting of halo, alkyl, haloalkyl, aralkyl, alkenyl, alkynyl, cycloalkyl, heterocyclyl, aryl, heteroaryl, heteroaralkyl, hydroxy, alkoxy, aryloxy, heteroaryloxy, amino, nitro, sulfhydryl, amido, acyl, carboxyl, oxycarbonyl, acyloxy, thioether, sulfonate, sulfonyl, sulfonamido, formyl, cyano and isocyano.

13. The compound of claim 12, wherein R is alkyl, alkenyl or alkynyl.

14. The compound of claim 13, wherein R is a C₁ to C₁₀ alkyl.

15. The compound of claim 14, wherein R is n-pentyl, iso-pentyl, neo-pentyl or t-pentyl.

16. A substantially pure and isolated compound of formula II: 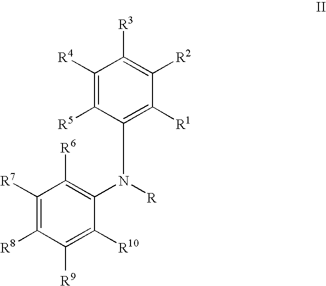
or a pharmaceutically acceptable salt thereof;
wherein, independently for each occurrence,
R is alkyl, alkenyl, alkynyl, aralkyl, or heteroaralkyl; and
R¹ to R¹⁰ are halo, azido, alkyl, aralkyl, alkenyl, alkynyl, cycloalkyl, heterocyclyl, aryl, heteroaryl, heteroaralkyl, hydroxy, alkoxy, aryloxy, heteroaryloxy, amino, alkylamino, arylamino, acylamino, heteroarylamino, nitro, sulfhydryl, imino, amido, phosphonate, phosphinate, acyl, carboxyl, oxycarbonyl, acyloxy, silyl, thioether, sulfonate, sulfonyl, sulfonamido, formyl, cyano or isocyano; wherein the aforementioned alkyl, alkenyl, alkynyl, cycloalkyl, heterocyclyl, aryl, heteroaryl, and heteroaralkyl may be optionally substituted with one or more groups selected from the group consisting halo, azido, alkyl, haloalkyl, fluoroalkyl, aralkyl, alkenyl, alkynyl, cycloalkyl, heterocyclyl, aryl, heteroaryl, heteroaralkyl, hydroxy, alkoxy, aryloxy, heteroaryloxy, amino, alkylamino, arylamino, acylamino, heteroarylamino, nitro, sulfhydryl, imino, amido, phosphonate, phosphinate, acyl, carboxyl, oxycarbonyl, acyloxy, silyl, thioether, sulfonate, sulfonyl, sulfonamido, formyl, cyano and isocyano.

17. The compound of claim 16, wherein R is alkyl or alkenyl aralkyl or heteroalkyl.

18. The compound of claim 17, wherein R is C₁-C₆ alkyl.

19. The compound of claim 18, wherein R is n-pentyl, isopentyl, neo-pentyl or t-pentyl.

20. The compound of claim 16, wherein at least one of R¹, R², R³, R³ or R⁵ is halo, alkyl, haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹; —N(R¹¹)₂ or —N(R¹¹)C(═O)R¹¹; and R¹¹ is hydrogen, or alkyl, haloalkyl, cycloalkyl, heterocycloalkyl, alkenyl, alkynyl, aryl, heteroaryl, aralkyl, or heteroaralkyl.

21. The compound of claim 20, wherein at least one of R¹, R², R³, R³ or R⁵ is —N(R¹¹)₂ or —N(R¹¹)C(═O)R¹¹.

22. The compound of claim 21, wherein at least one of R¹, R², R³, R³ or R⁵ is —N(R¹¹)₂.

23. The compound of claim 22, wherein R¹¹ is hydrogen.

24. The compound of claim 16, wherein at least one of R⁶, R⁷, R⁸, R⁹ or R¹⁰ is haloalkyl, aralkyl, alkenyl, alkynyl, cycloalkyl, heterocyclyl, aryl, heteroaryl, heteroaralkyl, —OR¹¹, —OC(═O)R¹¹, —SR¹¹, —S(═O)OR¹¹, —S(═O)₂OR¹¹, —S(═O)₂N(R¹¹)₂, —SC(═O)R¹¹, —N(R¹¹)₂ or —N(R¹¹)C(═O)R¹¹; and R¹¹ is hydrogen, or alkyl, haloalkyl, cycloalkyl, heterocycloalkyl, alkenyl, alkynyl, aryl, heteroaryl, aralkyl, or heteroaralkyl.

25. The compound of claim 24, wherein at least one of R⁶, R⁷, R⁸, R⁹ or R¹⁰ is —SR¹¹, —S(═O)OR¹¹, —S(═O)₂OR¹¹, —S(═O)₂N(R¹¹)₂, or —SC(═O)R¹¹.

26. The compound of claim 25, wherein at least one of R⁶, R⁷, R⁸, R⁹ or R¹⁰ is —SR¹¹.

27. The compound of claim 26, wherein —R¹¹ is hydrogen.

28. The compound of claim 16, wherein R¹⁰ is H.

29. The compound of claim 16, wherein R² is H.

30. The compound of claim 16, wherein R⁴ is H.

31. The compound of claim 16, wherein R⁵ is H.

32. The compound of claim 16, wherein R⁶ is H.

33. The compound of claim 16, wherein R⁷ is H.

34. The compound of claim 16, wherein R⁹ is H.

35. The compound of claim 16, wherein R¹⁰ is H.

36. A substantially pure and isolated compound represented by formula III: 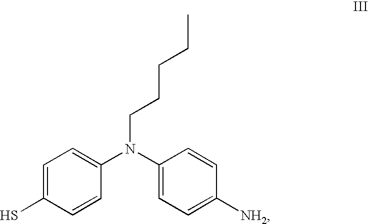
or a pharmaceutically acceptable salt thereof.

37. A pharmaceutical composition comprising a pure and isolated compound of claim 1 and a pharmaceutically acceptable carrier.

38. A method of treating or preventing a tryptase enzyme mediated condition in a subject in need thereof comprising administering to the subject an effective amount of a composition of claim 37.

39. The method of claim 38, wherein the tryptase enzyme mediated condition is an inflammatory or allergic condition.

40. The method of claim 39, wherein the tryptase enzyme mediated condition is allergic rhinitis, asthma, vascular injury, inflammatory bowel disease, psoriasis, arthritis, anaphylaxis, a wound, or an infection.

41. The method of claim 40, wherein the vascular injury is restenosis or atherosclerosis.

42. The method of claim 40, wherein the arthritis is rheumatoid arthritis, osteoarthritis or seronegative spondyloarthritis.

43. The method of claim 38, wherein the subject is a mammal.

44. The method of claim 43, wherein the subject is a primate.

45. The method of claim 44, wherein the subject is human.

46. A mixture comprising at least 10% of a compound claim 1.

47. The mixture of claim 46, wherein the compound comprises at least 25% of the mixture.

48. The mixture of claim 46, wherein the compound comprises at least 75% of the mixture.

49. The mixture of claim 46, wherein the compound comprises at least 95% of the mixture.
